(19) United States
(12) Patent Application Publication
(10) Pub. No.: US 2024/0401023 A1
(43) Pub. Date: Dec. 5, 2024

(54) OLIGONUCLEOTIDE EXTRACTION METHOD AND KIT

(71) Applicant: Waters Technologies Corporation, Milford, MA (US)

(72) Inventors: Mary Elizabeth Trudeau, Uxbridge, MA (US); Matthew A. Lauber, North Smithfield, RI (US); Nicole Lawrence, Stafford Springs, CT (US); Balasubrahmanyam Addepalli, Hopkinton, MA (US)

(73) Assignee: Waters Technologies Corporation, Milford, MA (US)

(21) Appl. No.: 18/679,628

(22) Filed: May 31, 2024

Related U.S. Application Data
(60) Provisional application No. 63/586,737, filed on Sep. 29, 2023.
(60) Provisional application No. 63/517,951, filed on Aug. 7, 2023.
(60) Provisional application No. 63/505,307, filed on May 31, 2023.

(51) Int. Cl. C12N 15/10; C12N 9/64

(52) U.S. Cl. CPC C12N 15/101; C12N 9/6424; C12Y 304/21064

(57) ABSTRACT

The present technology is directed to a method and kit for extracting oligonucleotides using solid phase extraction. Oligonucleotides are extracted from a biological sample by using a protease-assisted sample pretreatment step and a subsequent weak anion exchange solid phase extraction process.

### CROSS-REFERENCE TO RELATED APPLICATIONS

This application claims the benefit of U.S. Provisional Application No. 63/505,307 filed on May 31, 2023, U.S. Provisional Application No. 63/517,951 filed on Aug. 7, 2023, and U.S. Provisional Application No. 63/586,737, filed on Sep. 29, 2023, the entireties of which are incorporated herein by reference.

### FIELD OF THE TECHNOLOGY

The field of the present technology relates to methods and kits for performing solid phase extraction of oligonucleotides from a biological sample. The present technology achieves oligonucleotide extraction from the sample by using a protease-assisted sample pretreatment step and a subsequent weak anion exchange solid phase extraction process.

### BACKGROUND

Oligonucleotides are typically fragments of nucleic acids, such as intermediate degradation products of DNA and RNA or microRNAs, which regulate processes in biological systems. Their expression may be deregulated when diseases are developed. Therefore, oligonucleotides are proposed as a diagnostic and prognostic tool for various diseases. Oligonucleotides are also being developed as therapeutic drugs for a wide range of disease conditions. Accordingly, extraction of oligonucleotides from complex samples is useful in research and clinical diagnostic applications.

However, biological sample extraction of oligonucleotides from complex biological matrices such as plasma, blood, urine and tissue samples remain a formidable challenge in developing quantitative analytical methods for oligonucleotides. The polyanionic nature of oligonucleotides ensures that these compounds will be strongly bound to plasma proteins in addition to other matrix components. Successful bioanalytical sample preparation hinges on the difficult process of separating the oligonucleotide from the matrix.

### SUMMARY

The current technology employs a protocol and kit for extracting oligonucleotides from biological matrices that provides improved results as compared to other oligonucleotide extraction techniques, including protein precipitation, protein digestion, liquid-liquid extraction, reversed phase solid phase extraction (SPE), strong anion exchange SPE, or combinations thereof. Moreover, the present technology has unexpectedly superior recoveries and overcomes potential problems for applications with chromatography due to the large amount of matrix substances in solutions used for extraction.

The present technology includes methods and kit for performing solid phase extraction of oligonucleotides. Particularly, the present technology relates to a protocol for extracting one or more oligonucleotides from a biological sample that is in, one aspect, detergent free. In another aspect, the present technology uses a protease-assisted sample pretreatment step, and a subsequent weak anion exchange solid phase extraction process sorbent material comprising porous particles, wherein a surface of the porous particles is modified with a ligand bearing protonatable groups having a pKa value of between 7 and 12, preferably between 8 and 11, and more preferably between about 8 and about 10.

One of the objectives of the present technology is to provide a protocol and kit that needs no adjustments to be effective for the extraction of a wide range of modified oligonucleotides, including lipid modified oligos.

In accordance with some aspects, the present disclosure pertains to the combined use of a protease digestion that is directly combined with a weak anion exchange solid phase extraction.

In an aspect, the technology is directed to a method of extracting one or more oligonucleotides from a biological sample.

The method extraction may include one or more of the steps of (1) proteolytically digesting a sample, (2) loading the digested sample on a sorbent, (3) washing to remove any non-oligonucleotide components, and (4) releasing the desired oligonucleotides from the sorbent by flowing an elution solution through the sorbent, resulting in an eluate.

The above method steps may be achieved advantageously without using a detergent (i.e., “detergent free”) in the digestion mixture.

In a more particular aspect, the method steps include (1) proteolytically digesting the sample in a detergent free mixture by combining the biological sample with a protease solution, (2) loading the digested sample onto a sorbent material made up of porous particles, wherein a surface of the porous particles is modified with a ligand bearing protonatable groups having a pKa value of between 7 and 12, preferably between 8 and 11, and more preferably between about 8 and about 10, (3) flowing one or more washing solutions through the sorbent material such that one or more non-target components are removed from the sorbent material while the one or more target analytes are retained on the sorbent material, and (4) flowing an elution solution having a pH ranging from about 10 to about 12 though the sorbent material such that the retained one or more target analytes are released from the sorbent material into the elution solution (i.e., an eluate). In some examples, the protease solution is proteinase K. In some examples, the detergent free mixture future contains guanidine.

In an alternative aspect, the method steps include (1) proteolytically digesting the sample by combining the biological sample with a protease solution (e.g., proteinase K solution) and guanidine, (2) loading the digested sample onto a sorbent material comprising porous particles, wherein the surface of the porous particles is modified with a ligand bearing protonatable groups having a pKa value of between 7 and 12, preferably between 8 and 11, and more preferably between about 8 and about 10, (3) flowing one or more washing solutions through the sorbent material such that one or more non-target components are removed from the sorbent material while the one or more target analytes are retained on the sorbent material, and (4) flowing an elution solution having a pH ranging from about 10 to about 12 through the sorbent material such that the retained one or more target analytes are released from the sorbent material into the elution solution (i.e., an eluate).

The above aspects can include one or more of the following features. In some examples, the porous particles have a size greater than 5 μm and less than 100 μm.

In some examples, the method further includes subjecting the eluent solution to an analytical technique selected from liquid chromatography, mass spectrometry (MS), ultraviolet-visible spectroscopy, and combinations thereof.

In some examples, the one or more washing solutions comprises a partially aqueous organic solvent solution comprising methanol, ethanol, tetrahydrofuran (“THF”), acetonitrile, or combinations thereof.

In some examples, the elution solution comprises a base selected from organic amine, ammonium bicarbonate, ammonium hydroxide, ammonium acetate, or combinations thereof.

In some examples, the one or more elution solutions comprise the organic amine triethylamine (TEA). For example, 50 mM TEA in 50% MeOH. In some examples, the one or more elution solutions comprise a combination of TEA and ammonium bicarbonate, ammonium hydroxide, or ammonium acetate. In some examples, the one or more elution solutions comprise a combination of TEA and ammonium hydroxide. For example, the one or more elution solutions may be formed of 100 mM TEA in 50% MeOH with 0.30% NH₄OH or 50 mM TEA in 50% MeOH with 0.15% NH₄OH.

While the elution solution may comprise triethylamine as an organic amine, the technology is not limited to using TEA. For example, other organic amines can be utilized in elution solutions of the present technology. Other alternatives include, but are not limited to: dimethylamine, trimethylamine, ethanolamine, diethylamine, butylamine, dibutylamine, diisopropylamine, dimethylbutylamine, tripropylamine, diisopropylethylamine, hexylamine, octylamine, dicyclohexylamine, tributylamine, and dihexylamine. The organic amines can be combined with ammonium bicarbonate, ammonium hydroxide, or ammonium acetate to form the elution solution. In general, the concentration of the organic amine can be in the range of 2 to 500 mM, and more particularly between the range of 5 to 200 mM.

In some examples, the biological sample is a biological fluid. In preferred aspects, biological fluids include whole blood samples, blood plasma samples, serum samples, oral fluids, cerebrospinal fluids, fecal samples, nasal samples, and urine.

In some examples, the biological sample is a biological tissue. In preferred aspects, the biological tissue includes liver, kidney, and brain tissue, tissue homogenates, cells, and cell culture supernatants. In embodiments featuring tissue homogenates, a surfactant free tissue homogenization step can be performed prior to or simultaneously with proteolytically digesting the sample. When tissue homogenization occurs prior to digestion, a resulting tissue homogenate can be diluted 2 to 4 fold with an aqueous solution prior to incubation with a protease. In some embodiments, tissue homogenization includes applying an organic solvent with a concentration of greater than 20% volume (e.g., 50% volume or greater) of the biological sample.

In some examples, the one or more oligonucleotides are selected from double-stranded RNA, single-stranded RNA, single stranded DNA, double stranded DNA, double standard RNA/DNA hybrid, synthetic RNA, synthetic DNA, and combinations thereof, wherein the one or more nucleotides have a size ranging from 10 mer to 200 mer.

In some examples, the eluent solution further comprises an organic solvent at a concentration between 10 and 70% volume.

In some examples, the above methods may include diluting the elution solution comprising the one or more oligonucleotides with an equal volume of water.

In some examples, the technology is directed to a kit that contains the protease solution, the sorbent material, and the elution solution as described above.

The present technology and its related extraction protocols can also be used to facilitate the analysis of protein oligonucleotide conjugates. Through the use of the proteolytic digestion step, the oligonucleotide conjugate is converted into a peptide oligonucleotide conjugate. The peptide component becomes a surrogate to the larger protein component and the residual amino residues provide sequence localizing information. Ideally, the proteolysis generated peptide component is between 3 and 5 residues in length but it can also be between 1 and 20 residues in length. In the peptide oligonucleotide form, the SPE protocols described herein can be readily applied to effectively enrich the oligonucleotide analyte for sensitive LC-UV or LC-MS quantitation.

### BRIEF DESCRIPTION OF DRAWINGS

The technology will be more fully understood from the following detailed description taken in conjunction with the accompanying drawings, in which:

FIG. 1A shows a flow chart of an example of the method of extracting oligonucleotides.

FIG. 1B shows a flow chart of another method of extracting oligonucleotides in accordance with the present technology.

FIG. 2 shows oligonucleotide recovery across three samples using the present technology.

FIG. 3 shows the results of oligonucleotide plasma recovery using the present technology compared with using the results of the same sample with a comparative CLARITY® OTX™ oligonucleotide extraction protocol (Phenomenex, Inc. Torrance, CA).

FIG. 4 shows oligonucleotide IP-RPLC-MS direct inject sample eluate compatibility with minimal oligonucleotide (GEM 91) breakthrough and linear MS response with increased injection volume for samples prepared with eluate from the present technology (left) as compared to CLARITY® OTX™ (extraction protocol and products commercially available from Phenomenex, Inc.) eluate (right).

FIG. 5 shows oligonucleotide IP-RPLC-MS analytical performance (retention time, peak intensity, peak shape) using a LC-MS oligonucleotide performance standard mixture (non-extracted plasma sample) injected before (Injection #1) and after digestion pretreatment and SPE extraction of plasma containing the oligodeoxythymidine (15-35T), GEM 91, and GEM 132 oligonucleotides for the present technology (Injections #4 and #5).

FIG. 6 shows oligonucleotide IP-RPLC-MS analytical performance (retention time, peak intensity, peak shape) using a LC-MS oligonucleotide performance standard mixture (non-extracted plasma sample) injected before (Injection #1) and after digestion pretreatment and SPE extraction of plasma containing the oligodeoxythymidine (15-35T), GEM 91, and GEM 132 oligonucleotides for comparative CLARITY® OTX™ lysis pretreatment and SPE extraction (Injections #4 and #5).

FIG. 7 shows representative oligonucleotide IP-RP LC/MS system robustness, demonstrating stable MS % peak height response.

FIG. 8 shows consistent retention time for a protein digested oligo-deoxythymidine reference sample, 15-35T.

FIG. 9 shows flexible/scalable method performance by illustrating linear GEM 91 oligonucleotide MS response for digested and extracted plasma sample volumes from 12.5 to 300 μL.

FIG. 10 shows extracted oligonucleotide plasma sample cleanliness using targeted (MRM), and untargeted full scan TIC and EIC (total ion count and extracted ion count) IP-RP LC High Resolution MS analysis from the present technology.

FIG. 11 shows extracted oligonucleotide plasma sample cleanliness using targeted (MRM), and untargeted full scan TIC and EIC (total ion count and extracted ion count) IP-RP LC High Resolution MS analysis from the comparative CLARITY® OTX™ eluate.

FIG. 12 shows a comparative oligonucleotide plasma recovery example between CLARITY® OTX™ protocol and the present technology with modified SPE sample loading (top-loaded on water).

FIG. 13 shows comparative oligonucleotide recovery results of a spiked urine sample for three different elution solutions using the present technology with modified SPE sample loading.

FIG. 14 shows comparative oligonucleotide recovery results of a spiked plasma sample for three different elution solutions using the present technology with modified SPE sample loading.

### DETAILED DESCRIPTION

### Definitions

As used herein, the term “approximately” or “about” means +/−10% of the recited value.

As used herein, the term “includes” means includes but is not limited to, and the term “including” means including but not limited to.

As used herein, the term “sorbent,” or “sorbent material” refers to a material to which one or more components of the sample (e.g., oligonucleotides) adsorb. In some embodiments, the sorbent material of the present technology includes solid particles, preferably solid porous particles, e.g., solid silica, polymeric, or hybrid particles.

The terms “connected,” “conjugated,” “linked,” “attached,” “coupled,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used.

As used herein, the term “eluent” refers to a carrier portion of the mobile phase, such as a solvent or mixture of solvents with which a sample can be delivered in a chromatographic process.

As used herein, the term “eluate” refers to the material that emerges from or is eluted from a chromatographic process. To “elute” a molecule with an “eluent solution” (e.g., an oligonucleotide of interest or an impurity) from sorbent is meant to remove the molecule therefrom by altering the solution conditions such that buffer competes with the molecule of interest for binding to the sorbent. A non-limiting example is to elute a molecule from a sorbent by altering the pH of the buffer surrounding the sorbent.

As used herein the term “oligonucleotide” (or “OGN”) refers to a polymer sequence of two more nucleotides, including RNA, DNA, their analogs, including those having base modifications, sugar modifications or linkers used to modify the bioavailability. Oligonucleotides broadly include, but are not limited to, nucleotides from double-stranded RNA, single-stranded RNA, single stranded DNA, double stranded DNA, double standard RNA/DNA hybrid, synthetic RNA, synthetic DNA, and combinations thereof. Examples of OGN modifications include 2′-O-methoxyethyl, 2′-fluoro, phosphorothioate, and/or GalNAc modifications. Other examples of OGNs include antisense oligonucleotides (ASOs), small interfering RNAs (siRNAs), small hairpin RNAs (shRNAs), micro RNAs (miRNAs), messenger RNAs (mRNAs), and/or plasmids.

The size of “oligonucleotides” for the present technology are not limited; preferably, the one or more nucleotides have a size ranging from 10 mer to 200 mer.

As used herein, the term “stationary phase” refers to the phase or portion that is fixed in place or stationary in a chromatographic process, such as a solid material within a column through which the mobile phase passes.

As used herein, the term “mobile phase” refers to a phase or portion that moves in a chromatographic method, such as by passing through a column, and it includes the sample and the eluent.

As used herein, the term “functionalized,” “modified” or “chemically modified” refers to a changed state or structure of a molecule of this technology. Molecules may be modified in many ways including chemically, structurally, and functionally.

As used herein, the terms “extracting,” “separating,” or “isolating,” as used interchangeably herein, refer to increasing the degree of purity of a target molecule, i.e., one or more OGNs, a solution comprising the target molecule and one or more impurities. Typically, the degree of purity of the target molecule is increased by removing (completely or partially) an impurity from a composition.

As used herein, and unless stated otherwise, the term “sample” or “sample matrix” refers to any composition or mixture that contains one or more oligonucleotide(s) of interest. Samples may be derived from biological or other sources. Biological sources include eukaryotic and prokaryotic sources, such as plant and animal cells, tissues and organs. The sample may also include diluents, buffers, detergents, and contaminating species, debris and the like that are found mixed with the one or more oligonucleotide(s) of interest. The sample may be “partially purified” (i.e., having been subjected to one or more purification steps, such as filtration steps) or may be obtained directly from a host cell or organism producing the one or more oligonucleotide(s) of interest (e.g., the sample may comprise harvested cell culture fluid). For example, the sample matrix is clarified. That is, the sample has been subjected to a clarification step before the solid phase extraction. The sample matrix may be in the form of a solution. The solution may be heterogenous or homogenous.

As used herein, the term “detergent free” means the presence of protein solubilizing/cell membrane rupturing detergents (e.g., surfactants) such as in lysis buffers (e.g., TRITON™ X-100 (Polyoxyethylene octyl phenyl ether, commercially available from Dow Chemical Company) and TWEEN® 20 (Polyoxyethylene (20) sorbitan monolaurate, commercially available from Kroda Americas, LLC)) is absent from a composition or mixture as described herein.

Oligonucleotides (or OGNs) have a wide range of applications, including research, disease diagnosis, and therapy. OGNs used as therapeutics have high growth potential. They are used as starters in polymerase chain reactions, allowing for gene expression studies or probes for DNA sequencing, characterization, and tracking nucleic acids in biological systems. They are clinically tested as potential therapeutics in various diseases. Accordingly, there has been a significant breakthrough in this field in the past years.

Therefore, analysis of OGNs is important for impurity determination, degradation, or biotransformation product analysis. Medical utilization of OGNs also requires analysis, e.g., in disease diagnosis, while the quantitative and qualitative determination of oligonucleotides is a crucial aspect of clinical studies for their potential application as drugs.

One of the objectives of the present technology is to provide a protocol and kit that needs no adjustments to be effective for the extraction of a wide range of modified oligonucleotides, including lipid modified oligonucleotides.

The present technology pertains to the combined use of a protease digestion that is directly combined with a weak anion exchange solid phase extraction. That is, there is no intervening steps between the digestion and extraction.

The SPE sorbents of the present technology possesses high pH stability so that a protocol for elution can be performed with high pH (e.g., >9, preferably about 10 to about 12) eluents, preferably eluents with about 10 to about 12.

Another objective of the present technology is to improve the analyte recovery and/or the reproducibility of oligonucleotide extraction from complex biofluid matrices. The present technology allows selective extraction of oligonucleotides from complex biofluid matrices including tissue homogenates.

In an example, the present technology is directed to the preferential use of certain sorbent materials for performing solid phase extraction. In some examples, the SPE sorbent uses a weak anion exchange (WAX) mechanism. In a preferred example, the sorbent exhibits surface ligands a pKa between 8 and 11, and more preferably between about 8 and about 10.

In some examples, the SPE sorbent comprises a hydrophilic lipophilic balanced base particle, which ensures that hydrophobic modified oligonucleotides can be more readily eluted.

The sorbent material in accordance with this technology preferably contains porous particles.

The porous particles of the sorbent material are not limited in size. In preferred examples, a mean particle size of the porous particles is greater than 5 μm and less than 100 μm. The surface of the porous particles may be modified with a ligand bearing protonatable groups with a pKa value between 7 and 12, preferably between 8 and 11, and more preferably between about 8 and about 10.

The ion exchange capacity of the sorbents is not limited. In some examples, the ion exchange capacity of the sorbents is preferably between 0.1 and 1 milliequivalents of ion per gram of sorbents. In some examples, the ion exchange capacity is preferably between 0.2 and 0.8 milliequivalents per gram.

In another example, the present technology includes a solid phase extraction device or kit that contains the sorbent material of the present disclosure according to above aspects and embodiments. The kit may also contain a protease for rapidly digesting the protein composition of a sample prior to sample loading.

The specific protease of the present technology is not particularly limited as long as it is useful in proteolytical digesting biological samples and, preferably, without the use of a detergent. In some examples, the selected protease is proteinase K (e.g., T. album proteinase K).

In some examples, the proteolysis pretreatment step is incubated at above room temperature for greater than 5 min.

In some examples, the proteolysis pretreatment step involves the addition of a denaturant (e.g., guanidine) and the optional addition of a reducing agent. The number and kind of reducing agents are not limited. Some examples of reducing agents include dithiothreitol or tris(2-carboxyethyl) phosphine.

In some preferred examples, the proteinase K has a purity greater than 80%. In some preferred examples, the proteinase K has a purity greater than 90%.

In another preferred example, the proteinase K has an activity of at least 40 units per milligram of enzyme where one unit of proteinase K hydrolyzes urea-denatured hemoglobin producing color equivalent of 1 μmol tyrosine per 1 min at 37° C. and pH 7.5.

In another preferred example, the proteinase K is recombinantly expressed and purified from P. pastoris or E. coli.

In some examples, the nucleic acids of the OGNs have a size ranging from 10 mer to 200 mer. In some examples, the OGNs include N-acetyl galactosamine, modified and lipid modified single stranded and double stranded ribonucleic acid, and deoxyribonucleic acid oligomers or combinations thereof.

In another example, the present technology is directed to a method of pretreating a sample with a protease and subsequently performing solid phase extraction as follows: (1) mixing a sample with guanidine and a protease, (2) loading a sample fluid comprising one or more target oligonucleotides and a non-target component onto a sorbent material having a ligand with a pKa between 7 and 12, preferably between 8 and 11, and more preferably between about 8 and about 10; (3) flowing one or more washing solutions through the sorbent material, wherein the washing solutions remove a non-target component from the sorbent material while leaving target oligonucleotides retained on the sorbent material; and (4) flowing one or more about 8 to about 10 pH elution solutions though the sorbent material, wherein the target oligonucleotides retained on the sorbent material are released into one or more eluent solutions.

In some examples, after flowing the one or more elution solutions through the sorbent material, eluate from the elution will be subjected to analytical tools, techniques, or devices. Examples include chromatographic techniques such as liquid chromatography and detectors such as mass spectrometry (MS), ultraviolet-visible spectroscopy, or any combination thereof. In some examples, the eluate may be directly injected (i.e., without evaporation) into an ion pairing reversed phase separation.

In some examples, the one or more washing solutions comprises a partially aqueous organic solvent solution containing either methanol, ethanol, propanol, acetonitrile, or tetrahydrofuran.

In some examples, the one or more elution solutions have a pH ranging from 9 to 12.

In some examples, the one or more elution solutions comprises an anion such as a di, tri or tetravalent acid.

In some examples, the one or more elution solutions comprise one or more bases selected from an organic amine, ammonium bicarbonate, ammonium hydroxide, or ammonium acetate. In a preferred example, the one or more elution solutions comprises organic amine triethylamine (TEA).

The biological sample is not limited but should be the type of sample which can be proteolytic digested. In some examples, the sample contains biological fluids selected from whole blood samples, blood plasma samples, serum samples, oral fluids, cerebrospinal fluids, fecal samples, nasal samples, and urine. In some examples, the sample contains biological tissues such as liver, kidney, and brain tissue, tissue homogenates, cells, or cell culture supernatants.

In some examples, one or more OGNs include a double-stranded RNA, single-stranded RNA, single-stranded DNA, double-stranded DNA, double-stranded RNA/DNA hybrid, synthetic RNA, synthetic DNA or combination thereof.

The OGNs are not particularly limited in size. In some examples, OGNs have a size ranging from 10 mer to 200 mer.

In an example, the method of the present technology includes the steps of (1) proteolytically digesting the sample in a detergent free mixture by combining the biological sample with a protease solution, (2) loading the digested sample onto a sorbent material made up of porous particles having a surface of the porous particles that is modified with a ligand bearing protonatable groups having a pKa value of between 7 and 12, preferably between 8 and 11, and more preferably between about 8 and about 10, (3) flowing one or more washing solutions through the sorbent material such that one or more non-target components are removed from the sorbent material while the one or more target analytes are retained on the sorbent material, and (4) flowing an elution solution having a pH ranging from about 10 to about 12 through the sorbent material such that the retained one or more target analytes are released from the sorbent material into the elution solution (i.e., an eluate).

The resulting elution solution may be further diluted with water to optimize analysis. In a preferred example, the dilution is an equal volume of water to the resulting elution solution containing the released OGNs.

In some examples, the method may further comprise top-loading the digested sample to the SPE well containing water to improve OGN recovery and for diluting the amount of guanidine used in digestion. In a preferred example, the top-loading step is used to improve retention/binding of the OGNs containing a phosphodiester backbone to the SPE sorbent, subsequently improving oligonucleotide recovery.

In another example, the present technology is directed to a kit comprising the components described herein. In some examples, the components include the protease described herein, the SPE device described herein containing the sorbent (i.e., an SPE sorbent containing a ligand having a pKa between 7 and 12, preferably between 8 and 11, and more preferably between about 8 and about 10), and the SPE eluent described herein having a pH>9, preferably about 10 to about 12. In some examples, the SPE eluent may optionally contain an organic solvent at a concentration between 10 and 70% volume.

### EXAMPLES

### Example 1—Illustrated Example of Method of Extracting Oligonucleotides

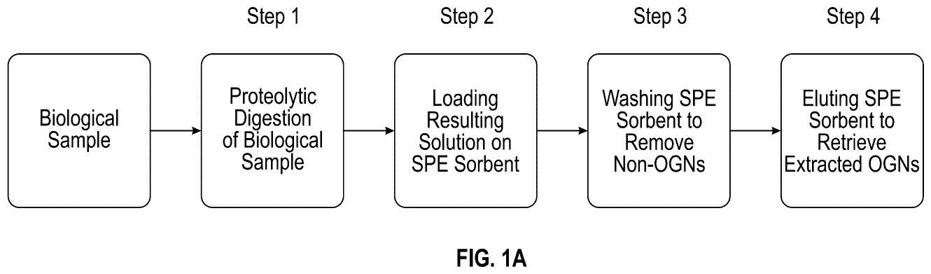

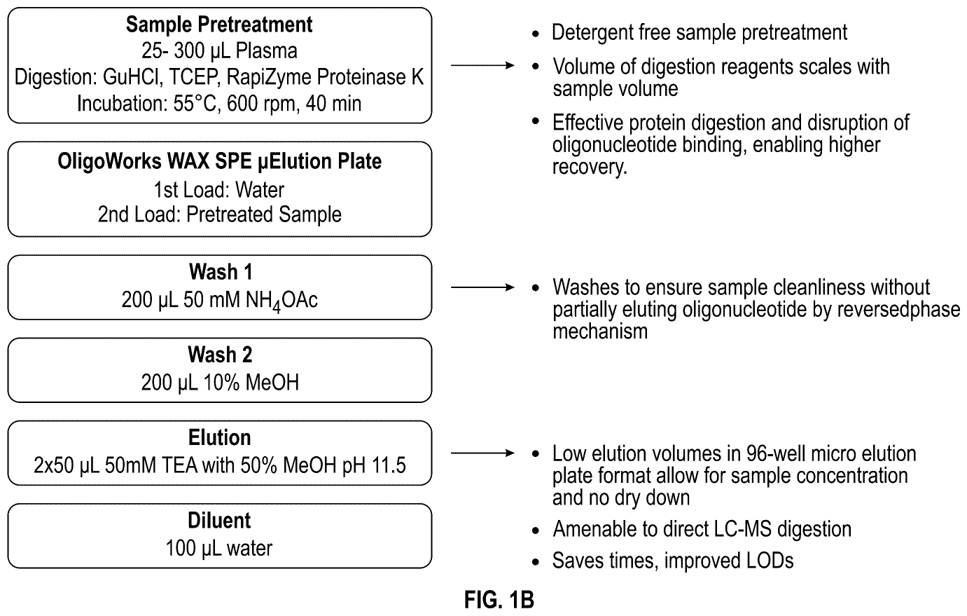

An illustrative example of the present technology is represented in a flow chart shown in FIG. 1A. As shown in FIG. 1A, four steps may be used to extract or isolate OGNs from a desired biological sample. The biological sample itself is not limited as long as it contains the desired OGNs that are to be extracted.

Step 1 is a pre-treatment step in which the sample is proteolytically digested using a protease. In this step, the sample is combined with a protease solution such as a proteinase K solution to form a digestion mixture.

In this example, the pre-treatment step also includes the addition of a reductant such as dithiothreitol or tris(2-carboxyethyl) phosphine. In this example, the pre-treatment step included dithiothreitol.

In some examples, the mixture may further contain guanidine (e.g., Guanidine HCl) for assisting with digestion of the sample matrix. Preferably and advantageously, the mixture is detergent free.

Following digestion, Step 2 is loading the digested sample onto a SPE sorbent material. The SPE sorbent material contains porous particles and uses a WAX mechanism as it is modified with a ligand bearing protonatable groups having a pKa value of between 7 and 12, preferably between 8 and 11, and more preferably between about 8 and about 10. Both OGNs and non-OGN components will be retained on the sorbent.

Following the loading step, Step 3 is flowing one or more washing solutions through the sorbent material such that one or more non-OGN components are removed from the sorbent material while the one or more desired OGNs are retained on the sorbent material.

Following the washing step, Step 4 is flowing the sorbent material with an elution solution having a pH ranging from about 10 to about 12 through the sorbent material such that the retained one or more OGNs are released from the sorbent material into the elution solution (i.e., an eluate).

While FIG. 1A illustrates an embodiment of an extraction process in accordance with the present technology, FIG. 1B illustrates another embodiment that is within the scope of the technology. Specifically, FIG. 1B illustrates a specific sample preparation and extraction protocol in accordance with the present technology. Sample pretreatment of a plasma sample is detergent free and includes proteolytically digesting the plasma sample by applying a proteinase K solution and guanidine. The plasma sample is incubated about 55° C., 600 rpm for 40 minutes. Next, the pretreated sample is loaded onto an elution plate containing weak anion exchange sorbent (Oligo Works WAX SPE u Elution plate, available from Waters Corporation, Milford, MA). To load the pretreated sample, water is first applied followed by loading of the pretreated sample. A first wash (200 μL of 50 mM NH₄OAc) and a second wash (200 μL of 10% MeOH) are applied to ensure sample cleanliness without partially eluting oligonucleotide by reversed-phase mechanism. Finally, elution is accomplished with 2×50 μL of 50 mM TEA with 50% MeOH at a pH of 11.5. The eluate can be diluted with 100 μL of water.

Without wishing to be bound by theory, it is believed that the pH of the SPE sorbent and elution solution is the driving mechanism for retention and elution of the desired OGNs. Combined with the pre-treatment step, this protocol provides superior extraction and analytical results over comparative technologies as will be further evidenced by the examples below.

### Example 2-Hydrophilic Lipophilic Polymer WAX SPE Used to Extract an Oligonucleotide from a Biofluid

Procedures were applied to extract several different modified oligonucleotides from a biofluid. A sample of this sort was created by mixing 100 μL rat plasma containing 1 μg/mL TRECOVIRSEN, which is GEM 91 (Gene Expression Modulator 91), GEM 132 (20 mer oligonucleotide targeting the HCMV UL36 gene), and Lipid Modified Oligo A. TRECOVIRSEN (GEM 91) is a 25-mer antisense oligodeoxynucleotide phosphorothioate molecule that targets HIV GAG RNA. GEM 132 is a fully phosphorothioated antisense oligonucleotide with 2′ methoxy caps that is used in the treatment of cytomegalovirus retinitis. Lipid modified oligo A is a negative control gapmer antisense oligonucleotide synthesized as shown below to have a 5′ palmitate modification, a phosphorothioate backbone, and terminal methoxy ethyl modifications. Lipid modifications are used with oligonucleotide therapeutics to increase their drug binding and cellular endocytosis.

### Lipid Modified Oligo A

100 μL of the spiked rat plasma was diluted with a mixture containing 20 μL of 6M guanidinium hydrochloride in 60 mM pH 7.5 Tris buffer (i.e., a non-lysis/detergent free buffer), 20 μL of 100 mM dithiothreitol (20 mM final) and 50 μL proteinase K 20 mg/mL solution (Qiagen, part number 19131). This mixture was vortexed and subsequently incubated at 65° C. for 15 minutes.

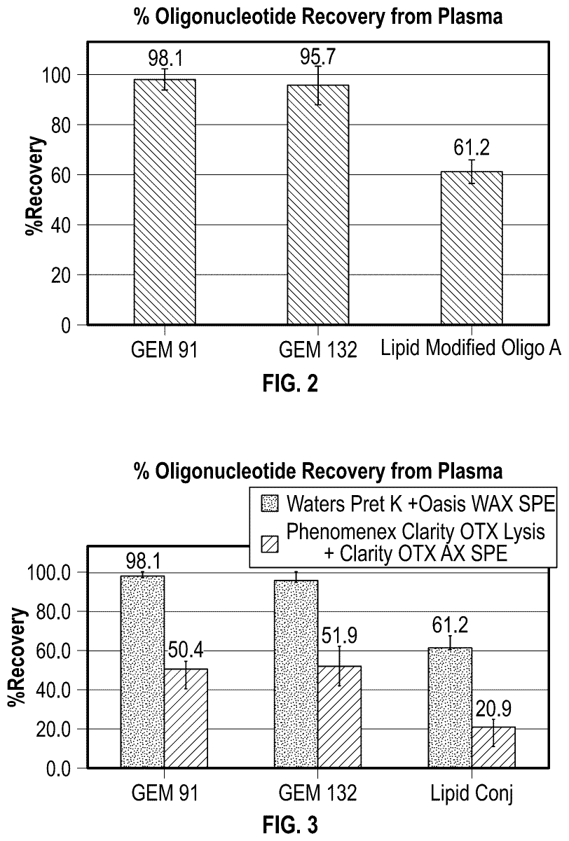

Solid phase extraction was next performed using a 96-well micro elution plate packed with 2 mg of OASIS® WAX 30 μm sorbent (a polymeric reversed phase, weak anion exchange mixed-mode sorbent commercially available from Waters Corporation, Milford, MA) and a vacuum manifold. Each sorbent bed was first conditioned with two 200 μL volumes of methanol and then equilibrated with two, 200 μL volumes of 50 mM ammonium acetate pH 5.5 buffer. To these conditioned beds, plasma digested samples were loaded and washed with another two 200 μL volumes of 50 mM ammonium acetate pH 5.5 buffer as well as one, 200 μL volume of 30% methanol. Finally, the adsorbed and purified analytes were eluted from the SPE wells using two, 50 μL volumes of an eluent comprised of 50 mM triethylamine in 50% methanol (pH 11.5). The resulting samples were diluted with an equal volume of water and then directly injected onto ion pairing reversed phase chromatography and detected by triple quadrupole mass spectrometry. Experimental conditions for performing these experiments are provided below. FIG. 2 demonstrates the recovery of each oligonucleotide.

TABLE 1
Instrument Method for IP-RPLC-MS

| | |
|---|---|
| UHPLC System | ACQUITY ™ PREMIER UPLC ™ |
| | (Waters Corporation) |
| Mobile phase A | 1% HFIP (Hexafluoro-2-propanol) |
| | 0.1% DIPEA (N,N- |
| | Diisopropylethylamine) in H₂O |
| Mobile phase B | 0.75% HFIP (Hexafluoro-2-propanol), |
| | 0.0375% DIPEA |
| | (N,N-Diisopropylethylamine, 65% ACN |
| | 35% H₂O |
| Column(s) | ACQUITY ® PREMIER ® Oligonucleotide |
| | BEH C18 1.7 um 2.1 mm × 50 mm |
| | (WATERS PN 186009484) |
| Col Temp | 50° C. |
| Sample Temp | 8° C. |
| Inj. Vol | 10 μL |
| Purge Solvent | 10:90 MeOH:H2O |
| Wash Solvent | 10:90 MeOH:H2O |
| MS | Xevo ® TQ-XS (Waters Corporation) |
| Capillary (kV) | 2.0 |
| Desolvation Temp | 500° C. |
| Desolvation flow | 1000 L/Hr |
| Cone Gas Flow | 150 L/Hr |

TABLE 2
IP-RP LC Gradient Parameters: 5 minute Analysis Time

| | Flow Rate | | | |
|---|---|---|---|---|
| Time (min) | (mL/min) | % A | % B | Curve |
| 0.0 | | 95 | 5 | 6 |
| 3.25 | 0.6 | 77 | 23 | 6 |
| 3.75 | 0.6 | 10 | 90 | 6 |
| 4.1 | 0.6 | 10 | 90 | 6 |
| 4.25 | 0.6 | 95 | 5 | 6 |

Table 1 shows the instrument method and protocol employed for IP-RPLC-MS. Table 2 shows the percentage recovery over a 5 minute run using the same flow rate. As can be seen, over 90% recovery occurs in less than 5 minutes.

As shown in FIG. 2, over 95% recovery was demonstrated for GEM 91 and GEM 132 samples and Lipid Modified Oligo A showed over 60% recovery, which is excellent given the difficulty of separating OGNs from the sample matrix with comparative methods that showed lower results.

### Example 3—Comparison Between Methods of Extraction of OGNs

Efficacy of the protocol of the present technology was tested and compared with alternative protocols. In particular, a comparative protocol based on CLARITY® OTX™ (a solid phase extraction protocol requiring a lysis-loading buffer and a mixed-mode, anion exchanger sorbent) was tested with the protocol of the instant technology using the same set of samples as described in Example 2. Surprisingly, and unlike comparative protocols, superior results were achieved without the need of detergent in the pre-treatment mixture.

CLARITY® OTX™ sample preparation solution relies on pretreating a biological sample containing one or more target oligonucleotides with a lysis-loading buffer and subsequently performing solid phase extraction as follows: (a) mixing a biological sample containing one or more target oligonucleotides 1:1 with the loading lysis buffer comprised of guanidine hydrochloride and TRITON™ X-100 (a detergent), (b) loading the mixed lysis buffer: biological oligonucleotide containing sample onto the AX sorbent material (CLARITY® OTX™, commercially available from Phenomenex, Inc.), (c) flowing 2 wash solutions through the sorbent material, wherein the washing solutions remove endogenous biological interferences from the sorbent material while leaving the target oligonucleotides retained on the sorbent material; and (c) flowing an elution solution comprised of (ammonium bicarbonate pH 9.5, acetonitrile, and tetrahydrofuran).

The primary components and extraction procedure of CLARITY® OTX™ are set forth in Tables 3 and 4 below. Other components present in the CLARITY® OTX™ lysis-loading buffer may include cysteine, TCEP, and sodium phosphate.

TABLE 3
Primary components in the CLARITY ® OTX ™ lysis-loading
buffer as reported in the manufacturer's safety data sheet

| Lysis Buffer Component | % |
|---|---|
| Guanidine Hydrochloride | 56-58 |
| TRITON ™ X-100 (detergent) | 1-3 |

TABLE 4
CLARITY ® OTX ™ AX SPE extraction procedure
using their AX sorbent from a biofluid

| Step | Description |
|---|---|
| Sample Pre- | Mix lysis buffer and oligo/serum in a 1:1 ratio, and |
| treatment: | vortex for 1 minute |
| SPE Sample | Load pretreated lysis:plasma sample |
| loading | |
| Wash 1: | 2 washes of ammonium acetate equilibration buffer |
| | (pH 5.5) |
| Wash 2: | 2 washes of ammonium acetate buffer (pH 5.5), |
| | containing acetonitrile |
| Elution: | Elution Solution Reagent: Ammonium Bicarbonate |
| | (pH 9.5), acetonitrile, and tetrahydrofuran |

FIG. 3 shows the results of oligonucleotide plasma recovery of Example 2 compared with using the results of the same sample with the CLARITY® OTX™ protocol. As can be seen in FIG. 3, for the sample, the protocol of the present technology provides about 2× improvement in oligonucleotide recovery from the GEM 91 and GEM 132 samples and 3× improvement for Lipid Modified Oligo A sample in comparison to the conventional protocol using a detergent (Lysis Buffer, i.e., TRITON™ X-100) and anionic exchange sorbent (i.e., the CLARITY® OTX™ protocol).

### Example 4—Demonstrated Oligonucleotide IP-RPLC-MS Method Robustness, Injection of Prepared and Extracted Biofluid Oligonucleotide Containing Samples Prepared with Protocol from Present Technology

An additional objective of the present technology is to ensure the prepared and extracted biological sample is amenable to IP-RPLC-MS analysis and can achieve robust LC-MS system performance, within and across days of analysis of extracted oligonucleotide plasma samples with LC column longevity (˜1000 injections).

With the present technology, the resulting sample eluate must have adequate cleanliness such that long-term, repeated LC-MS injections of these samples will not impede chromatographic separation and detection by UV or MS. For this, an oligonucleotide LC-MS performance standard mixture, containing LC-MS Gem 91, GEM 132 and oligodeoxythymidine reference standards were prepared and were injected on each day of analysis for the extracted plasma samples. The results were used as a measure of chromatographic and MS method performance across the multiple days of analysis.

Without being bound theory, the present technology, using an SPE extraction eluate containing methanol with weak eluotropic solvent strength, not only provides high oligonucleotide recovery, but can be directly injected on an LC-UV and/or MS instrument for analysis with linear response as injection volume increases and minimum oligonucleotide void-volume break-through. This is not the case with the CLARITY® OTX™ elution solution, which relies on the stronger eluotropic strength of acetonitrile and tetrahydrofuran.

Additionally, the eluate composition in the present disclosure facilitates direct sample injection of various injection volumes. This ability allows higher injection volumes of sample to increase oligonucleotide LC-MS detection and quantification, with little to no impact on LC-MS performance. This is particularly advantageous with sample preparation time vs the CLARITY® OTX™ solution. The stronger eluotropic solvent from Phenomenex's CLARITY® OTX™ product (i.e., acetonitrile and tetrahydrofuran) was severely restricted in injection volume due to the nature of its elution composition. This restriction limits, overall analytical method sensitivity and would require addition protocol steps to evaporation and reconstitute the sample in a more appropriate LC-MS amenable solution.

### Example 5—LC-MS Compatible Extraction Eluate

A sample was created by preparing a solution containing 1 μg/mL GEM 91 oligonucleotide and adding an equal volume of the present disclosure elution solution comprised of 50 mM triethylamine in 50% methanol (pH 11.5). A comparison sample was prepared by adding an equal volume of the 1 μg/mL GEM 91 oligonucleotide solution to the CLARITY® OTX™ elution solution comprised of 100 mM ammonium bicarbonate, pH 9.5, 40% acetonitrile, and 10% tetrahydrofuran. The resulting representative oligonucleotide containing elution solutions (i.e., the sample of the present disclosure and the comparison sample) were diluted 1:1 with water and were then directly injected onto ion pairing reversed phase chromatography and detected by triple quadrupole mass spectrometry, using injection volumes of 2.5, 5 and 10 μL.

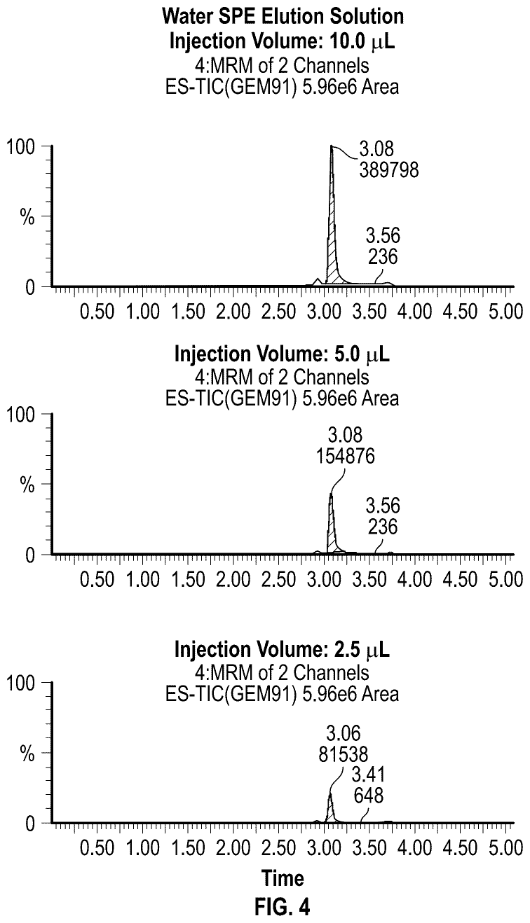

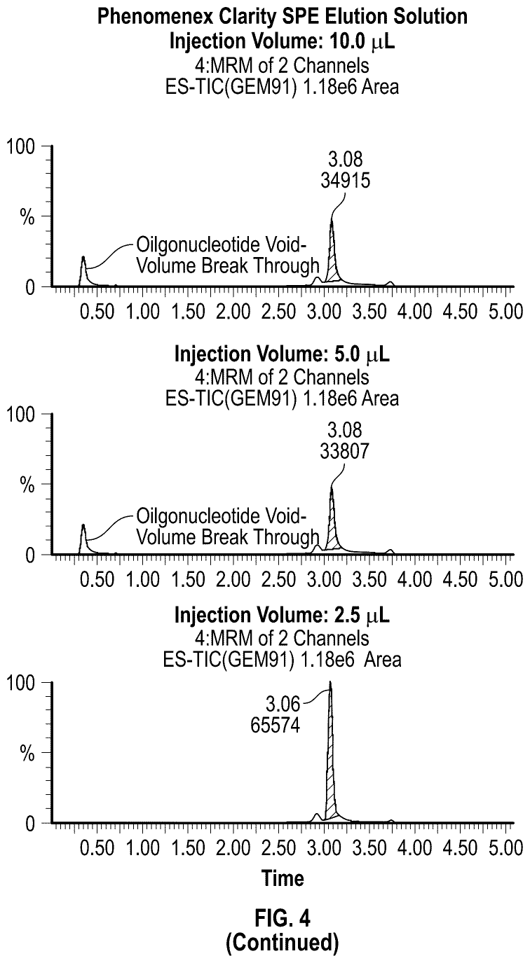

FIG. 4 demonstrates oligonucleotide IP-RPLC-MS direct inject sample eluate compatibility with minimal oligonucleotide (GEM 91) breakthrough and linear MS response with increased injection volume for samples prepared with eluate from the present technology (left) as compared to the comparison sample utilizing the CLARITY® OTX™ eluate (right) showing significant oligonucleotide void volume break-through and decreased oligonucleotide response with increased injection volume.

### Example 6—Consistent LC-MS System Performance Injecting Prepared and Extracted Plasma Samples

A LC-MS oligonucleotide performance standard mixture was created using the current technology (Example 2) and comparative CLARITY® OTX protocols. The resulting LC-MS oligonucleotide performance standard and extracted plasma samples were then directly injected (10 μL) onto an ion pairing reversed phase chromatography and detected by triple quadrupole mass spectrometry.

TABLE 5
Mass Spectrometer Multiple Reaction Monitor
(MRM) Oligonucleotide Transitions for the
Oligodeoxythymidine Reference Stand

| Oligodeoxy- | | | | |
|---|---|---|---|---|
| thymidine | | | | |
| Reference | Parent | Daughter | Cone | Collision |
| Standard (OST) | (m/z) | (m/z) | (V) | (eV ) |
| OST 15T | 642.00 | 303.00 | 50 | 25 |
| OST 20T | 646.40 | 303.00 | 50 | 25 |
| OST 25T | 668.10 | 303.00 | 50 | 25 |
| OST 30T | 684.70 | 303.00 | 50 | 25 |
| OST 35T | 704.60 | 303.00 | 50 | 25 |

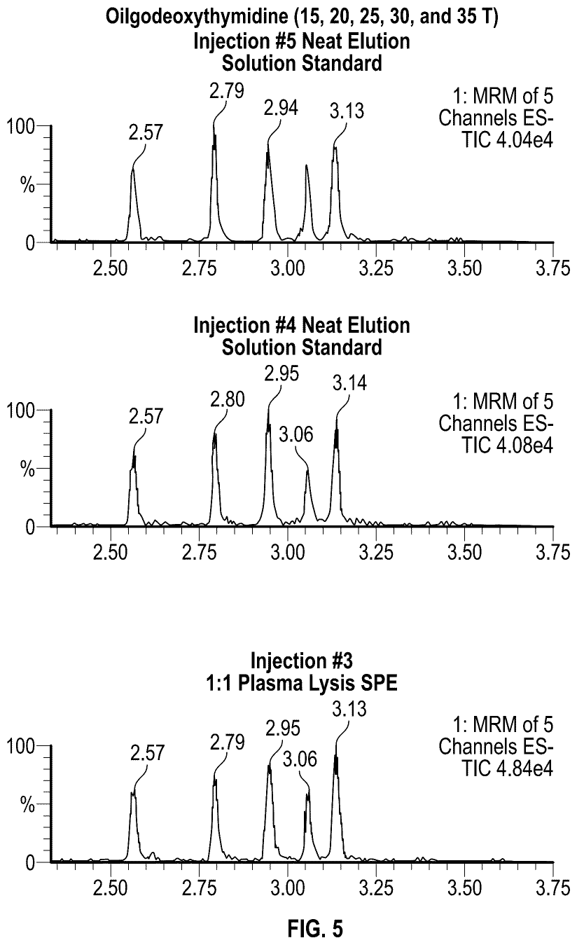

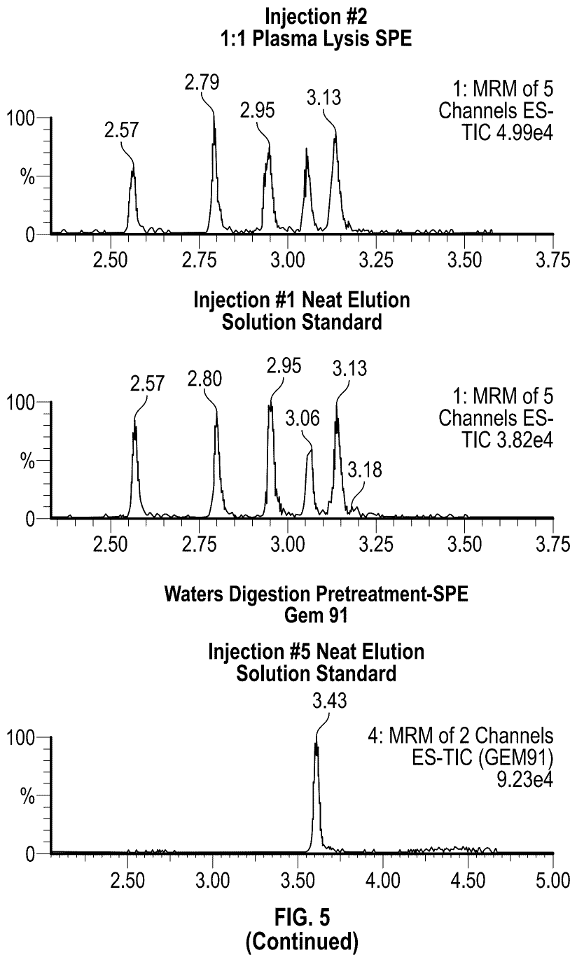

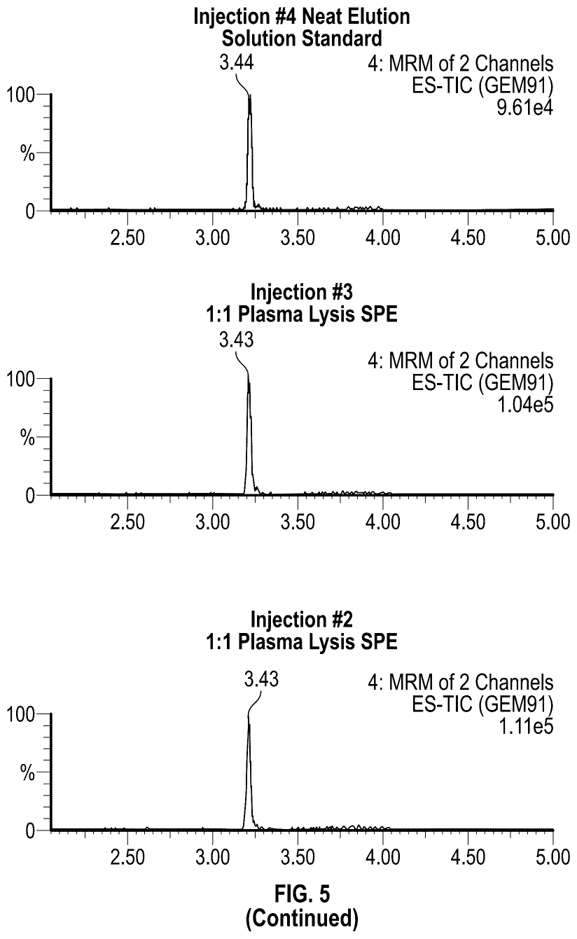

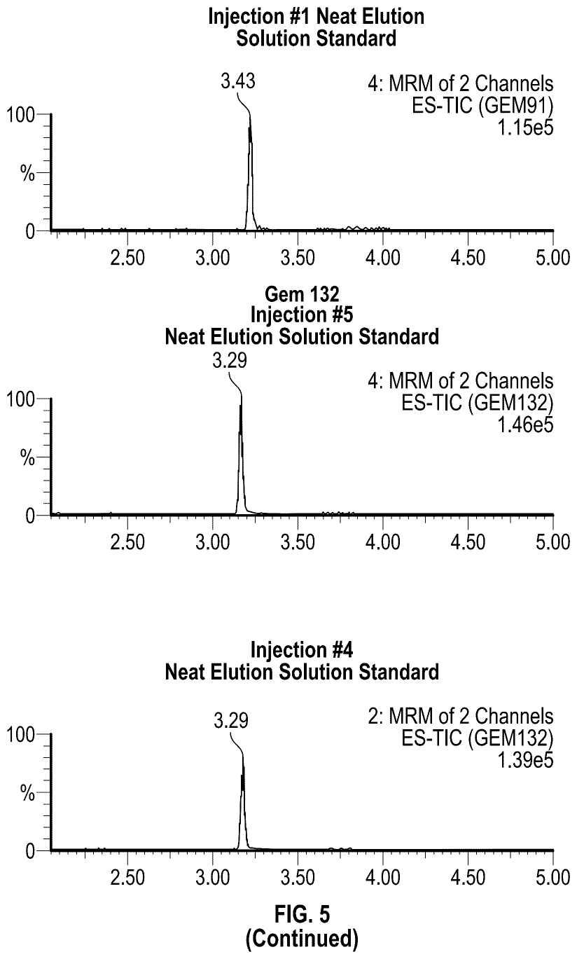

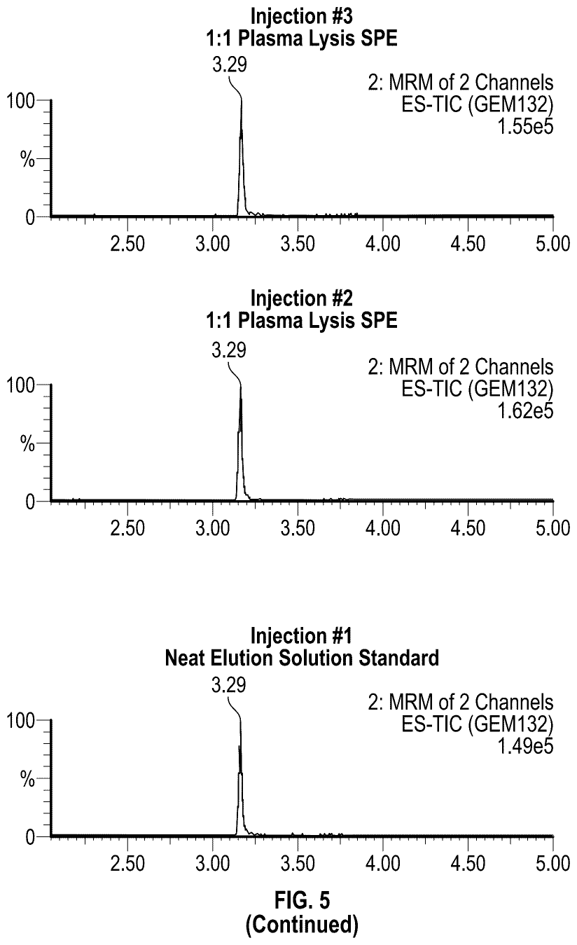

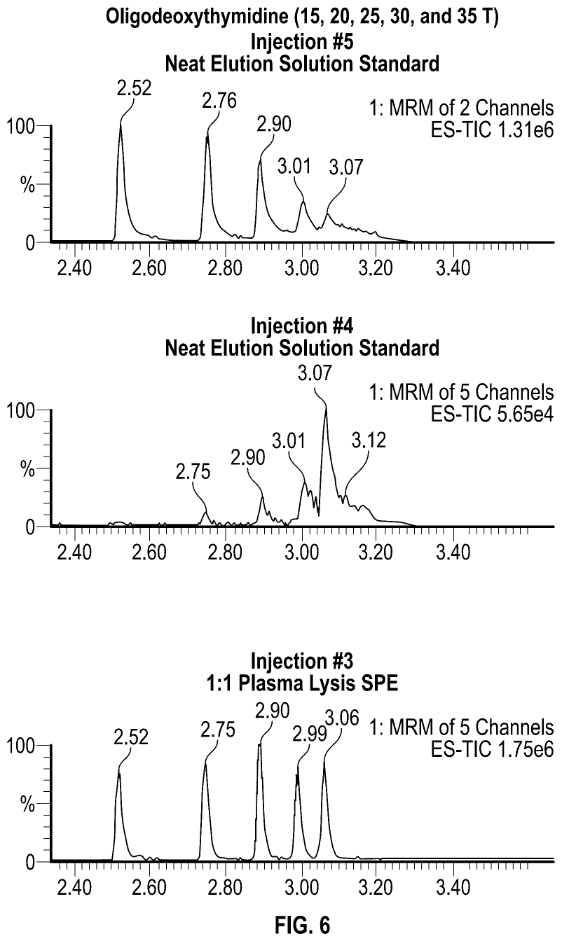

FIGS. 5 and 6 show oligonucleotide IP-RPLC-MS analytical performance (retention time, peak intensity, peak shape) using a LC-MS oligonucleotide performance standard mixture (non-extracted plasma sample) injected before and after digestion pretreatment and SPE extraction of plasma containing the oligodeoxythymidine (15-35T), GEM 91, and GEM 132 oligonucleotides for present disclosure (FIG. 5) as compared to CLARITY® OTX™ lysis pretreatment and SPE extraction (FIG. 6) showing significant oligonucleotide peak distortion and signal loss for the neat elution solution standard injected standards (#4 and #5).

### Example 7—Consistent LC-MS System Performance Column Longevity

A LC-MS oligonucleotide performance standard mixture was made using the current technology (Example 2) and comparative CLARITY® OTX™ extraction protocols. These representative LC-MS performance standard mixture samples (i.e., the sample in accordance with the present technology, and the comparative sample) were injected prior to and after injection of prepared and extracted oligonucleotide containing plasma samples. The plasma samples (100 μL) containing 1 μg/mL of GEM 91, 1 μg/mL of GEM 132 were prepared and extracted as described using the protocol in accordance with Example 2 (the present technology), or using the CLARITY® OTX™ protocol. The resulting LC-MS oligonucleotide performance standard and extracted plasma samples were then directly injected (between 2-10 μL) onto an ion pairing reversed phase chromatography and detected by triple quadrupole mass spectrometry. Samples are prepared and analyzed on a minimum of 5 separate days, injecting ˜175 extracted plasma samples with the LC-MS oligonucleotide performance standard injected at the beginning, middle and end of the run for each day. Experimental conditions for performing these experiments were provided in the examples above.

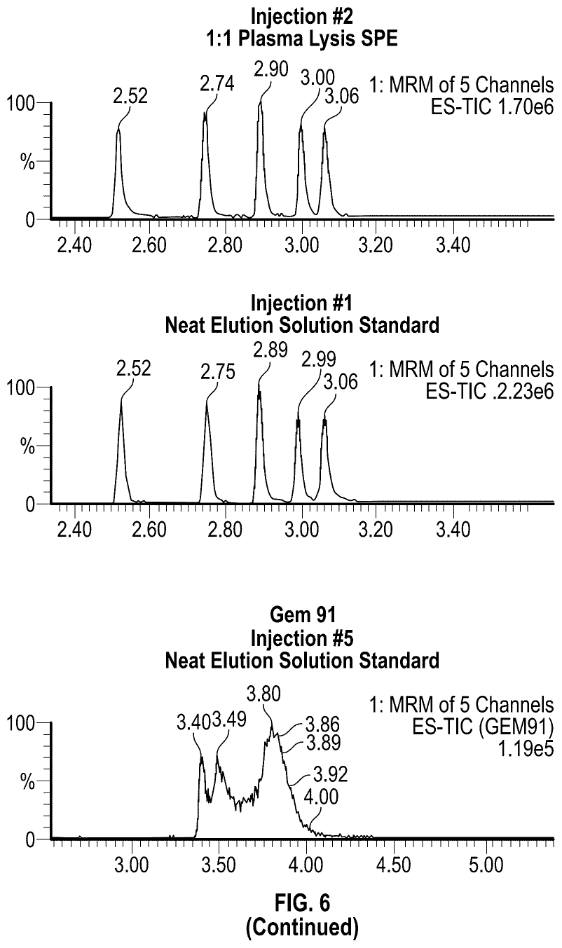

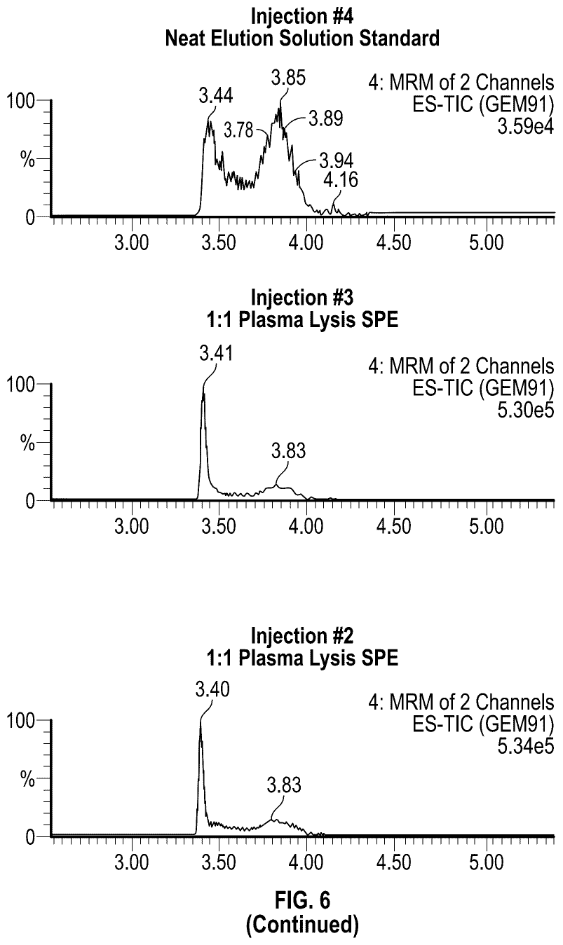

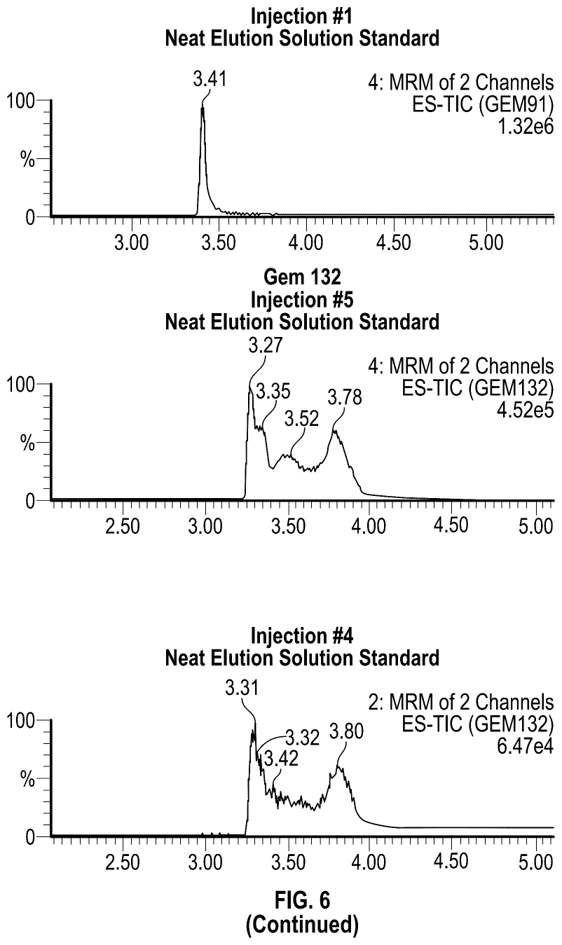

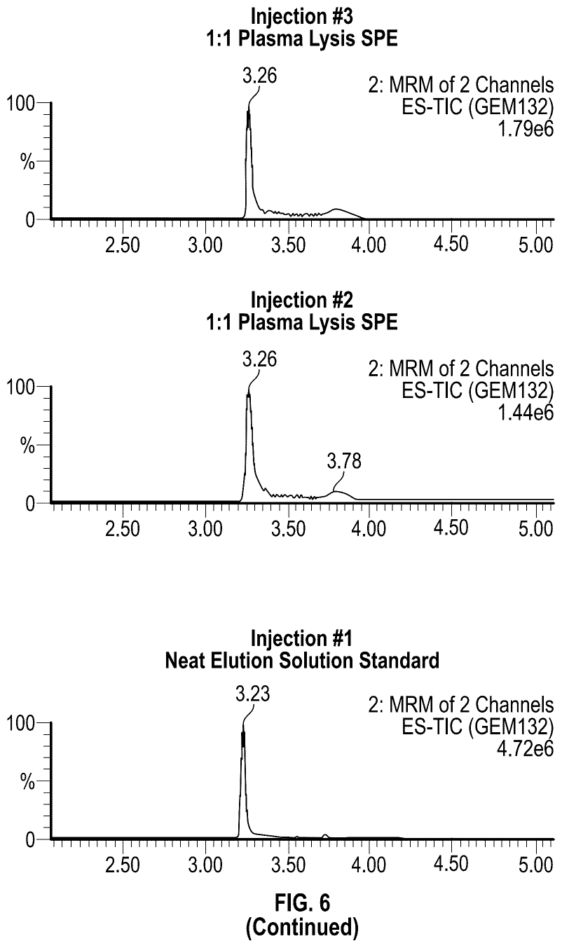

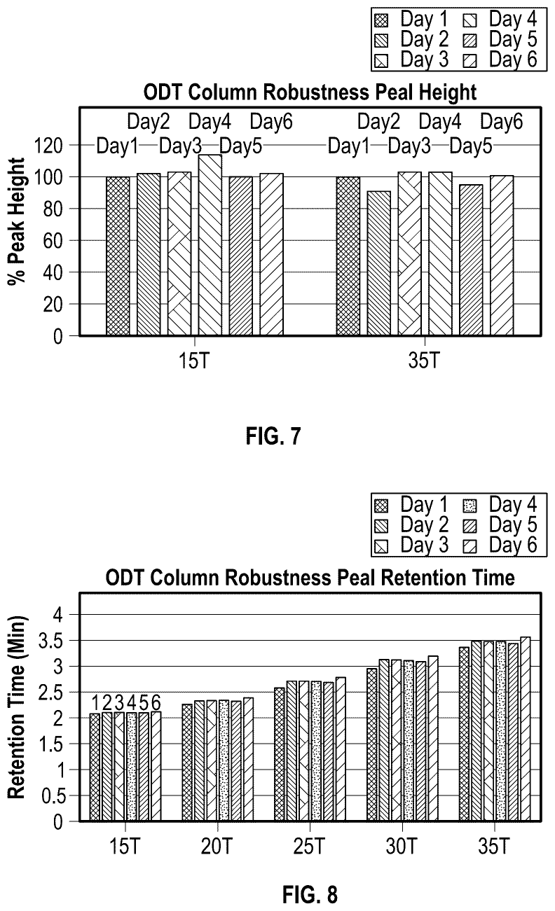

FIGS. 7 and 8 show representative oligonucleotide IP-RP LC/MS system robustness, demonstrating stable MS % peak height response (FIG. 7) and consistent retention time for the MASSPREP® (a protein digestion standard mixture, commercially available from Waters Corporation, Milford, MA) oligo-deoxythymidine reference sample, 15-35T (FIG. 8) injected across six days of analysis for the protease digested and SPE extracted samples, in accordance with the present application, with column longevity (>950 plasma extracted samples).

### Example 8—Demonstrated Flexible and Scalable Extraction Performance

A LC-MS oligonucleotide performance standard mixture was made using the current technology (Example 2) and comparative CLARITY® OTX™ protocols. The resulting LC-MS oligonucleotide performance standard and extracted plasma samples are then directly injected (between 2-10 μL) onto an ion pairing reversed phase chromatography and detected by triple quadrupole mass spectrometry. Samples were prepared and analyzed on a minimum of 5 separate days, injecting ˜175 extracted plasma samples with the LC-MS oligonucleotide performance standard injected at the beginning, middle and end of the run for each day. Experimental conditions for performing these experiments were provided in the examples above. New LC-columns are employed for the extracted plasma sets from the present technology and for the extracted plasma sample sets created using the CLARITY® OTX™ protocol.

Plasma samples from 12.5 to 300 μL containing 1 μg/mL of GEM 91, 1 μg/mL of GEM 132 were prepared and extracted as described using the protocol in Example 2, with modification to the sample digestion step and extracted using the micro-elution 96-well SPE format. LC-MS oligonucleotide performance standard and extracted plasma samples were then directly injected (10 μL) onto an ion pairing reversed phase chromatography and detected by triple quadrupole mass spectrometry.

The results are presented in Table 6 below.

TABLE 6
Plasma and digestion reagent volumes used to assess plasma
digestion pretreatment scalability and extraction performance

| Starting | | | | |
|---|---|---|---|---|
| Oligonucleotide | | | | Final |
| containing | Guanidine | Dithiothreitol | Proteinase | Digested |
| plasma | Volume | Volume | K | Sample |
| volume (μL) | (μL) | (μL) | (μL) | (μL) |
| 12.5 | 1.25 | 1.25 | 2.5 | 17.5 |
| 25.0 | 2.50 | 2.50 | 5.0 | 35 |
| 50.0 | 5.0 | 5.0 | 10.0 | 70 |
| 100.0 | 10.0 | 10.0 | 20.0 | 140 |
| 200.0 | 20.0 | 20.0 | 30.0 | 250 |
| 300.0 | 30.0 | 30.0 | 40.0 | 400 |

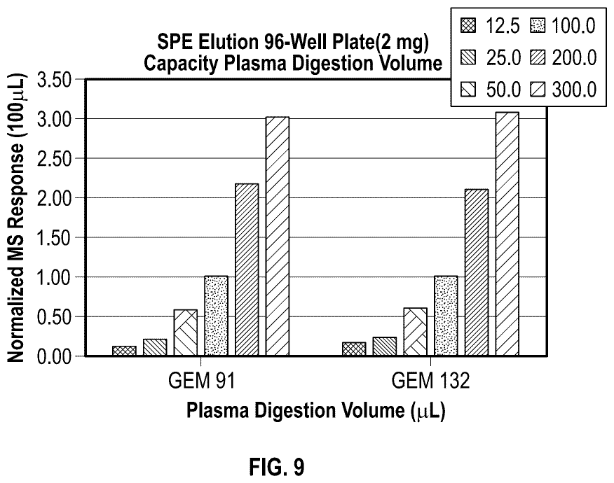

FIG. 9 shows flexible/scalable method performance by illustrating linear GEM 91 oligonucleotide MS response for digested and extracted plasma sample volumes from 12.5 to 300 μL.

### Example 9—Minimal MS Matrix Artifacts Resulting from Both Biological Matrix and Reagent Artifacts in the Extracted Eluate Sample

An additional objective of the present technology is to ensure the prepared and extracted biological sample, prepared as described in this disclosure, is sufficiently clean and amenable to IP-RPLC-MS, such that there are minimal MS matrix artifacts resulting from both biological matrix and reagent artifacts in the extracted eluate sample.

Without being bound by theory, the protocol of the instant technology allows the elution of the oligonucleotide analytes with an eluent comprised of a comparatively weak eluotropic strength solvent. In one example, the eluent contains 50% (v/v) methanol. This ensures that reasonably high injection volumes can be directly injected onto an ion pairing reversed phase separation without breakthrough. A 2-fold water dilution of the eluate can be performed to further facilitate high volume loads. In turn, evaporation of the eluate can be avoided for faster turnarounds. The acetonitrile and THE content of the CLARITY® OTX™ eluate has a significantly stronger solvent eluotropic strength that compromises direct injection.

With the present technology, directed to a sample preparation method of pretreating a biological fluid oligonucleotide containing sample with a protease followed by solid phase extraction, the resulting sample eluate has adequate cleanliness, minimizing matrix interferences derived from both the sample pretreatment and extraction reagents, as well as the biological matrices. Reducing these interferences facilitates increased selectivity and specificity of the overall method.

The plasma samples (100 μL) containing 0.1-5 μg/mL of GEM 91, 1 μg/mL of GEM 132 are prepared and extracted as described using the protocol of Example 2 or using the CLARITY® OTX™ protocol. The resulting LC-MS oligonucleotide performance standard and extracted plasma samples were then directly injected (between 2-10 μL) onto an ion pairing reversed phase chromatography and detected by triple quadrupole and HRMS mass spectrometry. Experimental conditions for performing these experiments were provided in the examples above.

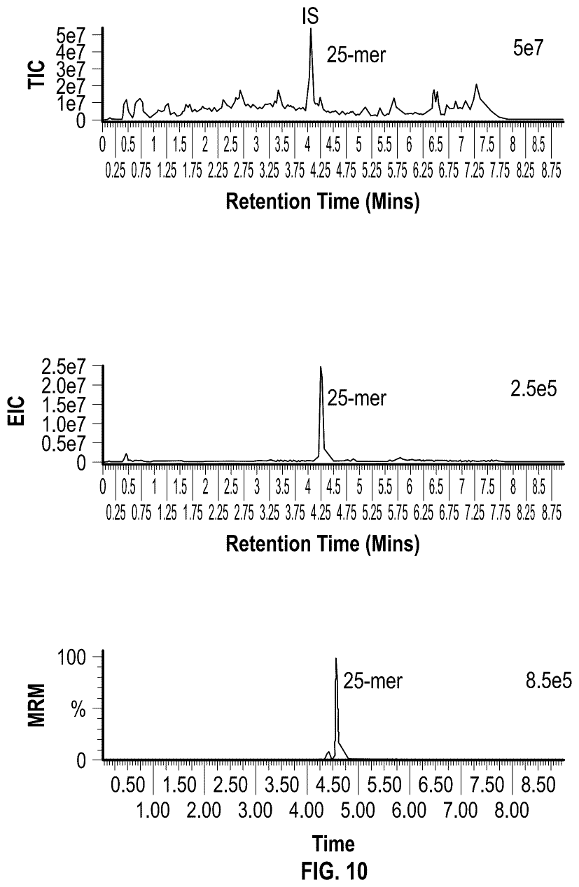

Example comparison of extracted oligonucleotide plasma sample cleanliness using targeted (MRM), and untargeted full scan TIC and EIC (total ion count and extracted ion count) IP-RP LC High Resolution MS analysis from present disclosure (FIG. 10) as compared to CLARITY® OTX™ eluate (FIG. 11) showing significant oligonucleotide void volume break-through and decreased oligonucleotide response with increased injection volume.

### Example 10—Improved Oligonucleotide Recovery as Compared to CLARITY® OTX™ Showing an Increase in Recovery of Phosphodiester Backbone Containing Oligonucleotides when Loaded to SPE Well Top-Loaded with Water

An additional objective of the present technology is to ensure the prepared and extracted biological sample, prepared as described in this disclosure, is sufficiently clean and amenable to IP-RPLC-MS, such that there are minimal MS matrix artifacts resulting from both biological matrix and reagent artifacts in the extracted eluate sample. To meet the objective of high oligonucleotide recovery for a diversity of oligonucleotides from extracted biological sample, efforts were focused on ensuring complete digestion of the plasma sample and proper SPE sample loading and elution conditions to achieve it. It was observed that SPE recovery of oligonucleotides containing a phosphodiester backbone were improved when the described SPE protocol in the examples above were modified by top-loading the digested sample to an SPE well containing water.

Without being bound by theory, it is believed that the Guanidine HCl reagent used in the digestion of the sample, interferes with oligonucleotide binding to the weak anion exchange sorbent, Thus, diluting the digested sample with water upon SPE loading reduces the guanidine concentration. This reduction in concentration improves the binding or retention of the oligonucleotide to the sorbent, thus yielding higher oligonucleotide recovery.

The example was prepared as follows: to rat plasma, a standard mix of oligodeoxythimidine (15, 20, 25, 30 and 35 mer) and ssDNA (20 mer) were added to yield final concentrations of 0.01 μmol/uL (Oligodeoxythymidine) and 1 ug/mL of the ssDNA.

100 μL of the oligodeoxythimidine (15-35 NT) mer and ssDNA containing rat plasma was diluted with 20 μL of 6M guanidinium hydrochloride in 60 mM pH 7.5 Tris buffer (0.66 M final), 20 μL of 300 mM tris(2-carboxyethyl) phosphine (30 mM final) and 50 μL proteinase K, 20 mg/mL solution (Qiagen, part number 19131). This mixture was vortexed and subsequently incubated at 55° C. for 60 minutes.

Solid phase extraction was next performed using a 96-well micro elution plate packed with 2 mg of OASIS® WAX 30 μm sorbent (a polymeric reversed-phase, weak anion exchange mixed-mode sorbent commercially available from Waters Corporation, Milford, MA) and a vacuum manifold. Each sorbent bed was first conditioned with two 200 μL volumes of methanol and then equilibrated with two, 200 μL volumes of 50 mM ammonium acetate pH 5.5 buffer. The entire volume of the digested plasma samples (190 μL) were either loaded directly to the conditioned plate or loaded on-top of 100 μL water pre-loaded to the SPE wells. These samples were then slowly evacuated from the SPE wells. The water top-load reduced the guanidine concentration in the digested plasma sample from 0.63 M to 0.41 M final upon SPE loading. The samples were then washed with another two 200 μL volumes of 50 mM ammonium acetate pH 5.5 buffer as well as one, 200 μL volume of 10% methanol. Finally, the adsorbed and purified analytes were eluted from the SPE wells using two, 50 μL volumes of an eluent comprised of 50 mM triethylamine in 50% methanol (pH 11.5). The resulting samples were diluted with an equal volume of water and then directly injected onto ion pairing reversed phase chromatography and detected by triple quadrupole mass spectrometry.

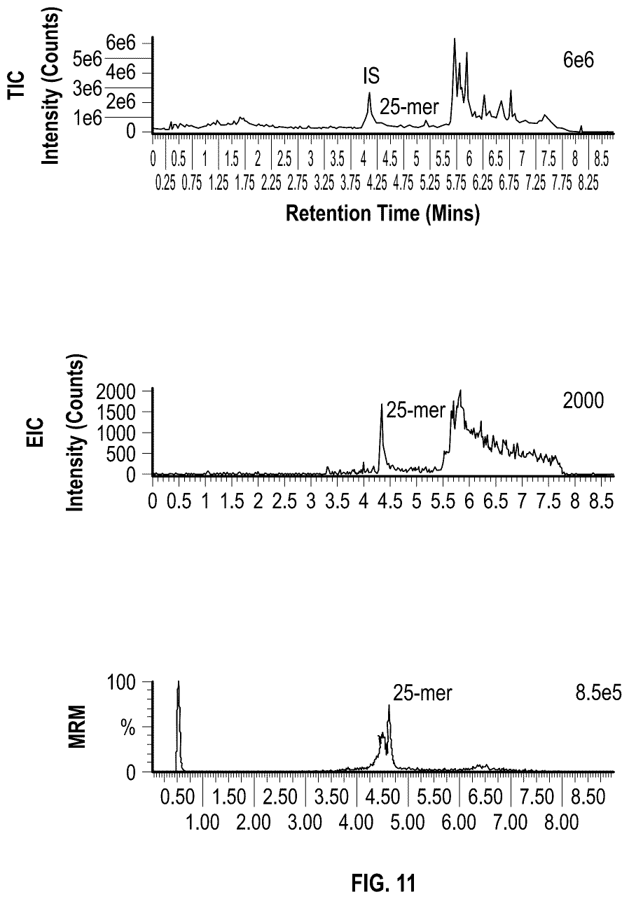

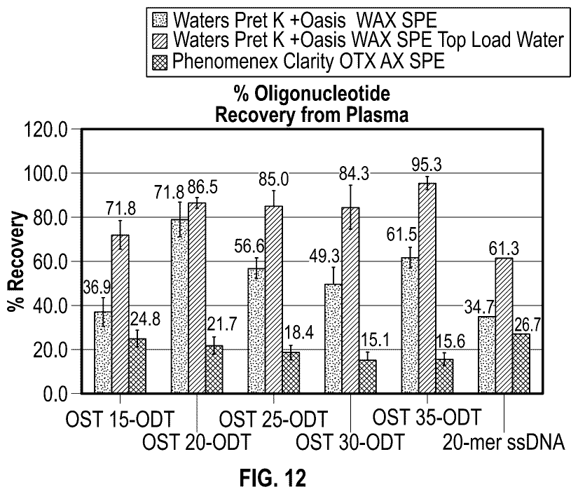

FIG. 12 demonstrates the recovery of each oligonucleotide using the sample preparation and extraction presented in the examples above, using direct plasma sample SPE loading and top-loaded onto and SPE well containing water, and CLARITY® OTX™ protocol results.

As can be seen in FIG. 12, top-loading vastly improved recovery of the method used in the above examples and more than 2× over the comparative CLARITY® OTX™ protocol.

### Example 11—Using a Detergent Free Protocol with a Biological Sample Formed from a Tissue Homogenate

The extraction protocols of the present technology can be utilized with organ tissues. To create a sample, the organ tissue is homogenized prior to or simultaneously with the proteolytic digestion of protein matter. The following example illustrates an embodiment in which digestion occurs after homogenization.

Thirty (30) mg of organ tissue is added to zirconia, ceramic, glass or steel beads along with 70 μL methanol, 20 μL 6M guanidine denaturant solution, and 10 μL 0.5M TCEP reducing agent solution. Lysis is then performed by bead disruption using a tissue homogenizer (Precellys homogenizer, Bertin Corporation, Rockville Maryland). A solution with this composition can also be used with sonication and pressure/shear force-based disruption. In this step, it is advantageous to have a tissue weight to solvent volume of greater than 1 mg to 2 μL solvent but less than 1 mg to 10 μL solvent. Guanidine solution is optionally added from a stock solution comprised of 3 to 6 M guanidine. The guanidine counter ion can be, but is not limited to, chloride or thiocyanate. The organic solvent can be methanol or another protic or aprotic solvent. Methanol and ethanol are preferred. For bead-based disruption, this procedure can be performed with homogenization beads ranging in diameter from 0.1 to 2 mm.

Upon collecting the homogenate, 50 μL of water is added to the tube followed by 50 μL 20 mg/mL proteinase K. The sample is incubated for 40 min at 55° C., or 1 hr at 40° C. if a duplex oligonucleotide is to be analyzed. The proteinase K concentration and aliquot volume can be optimized along with the time and temperature of the incubation to achieve a desired completeness of digestion.

In an alternative embodiment, the proteinase K is added to the tissue sample before or during the homogenization step (i.e., digestion is simultaneous with homogenization). In some embodiments, a proteolytic enzyme different than proteinase K is applied.

In an optional protocol step, the proteinase K digested tissue homogenate can be clarified of particulate matter with procedures including but not limited to a high-speed centrifugal pelleting or filtration step. Supernatant or filtered solution is to be further processed by solid phase extraction techniques outlined above (e.g., FIG. 1A or FIG. 1B).

### Example 12—Comparison of Elution Solutions for Use with Oligonucleotide Solid Phase Extraction Method

In this example, three different elution solutions (i.e., eluents) were used in a solid phase extraction method in accordance with the present technology and recovery of the oligonucleotide extraction was compared. The three eluents each contained triethylamine (TEA), and some of the eluants also included ammonium hydroxide. Specifically, eluent 1 included 50 mM TEA, 50% MeOH. Eluent 2 included 50 mM TEA, 50% MeOH, 0.15% NH₄OH. And eluent 3 included 100 mM TEA, 50% MeOH, 0.3% NH₄OH.

To compare recovery results based on elution solution type (i.e., eluent 1, eluent 2, or eluent 3), a stock sample was prepared as follows: to a lyophilized sample of 20-mer ssDNA Promoter (10 μg, 1.6 nmol) the following components were added and mixed: 1.74 μL Tyrosine (Tyr, 10 mg/mL) (9.6 nmol); 100 μL 100% MeOH; 17.5 μl Milli-Q water; and 2000 μL of 200 mM ammonium acetate pH5.5 to reach a final volume of 4000 μL.

The prepared stock sample was then subjected to solid phase extraction technique using a 10 mg 1 cc cartridge using the following protocol. The cartridge was conditioned using 1000 μL of 100% MeOH to run through the cartridge to waste. The conditioned cartridge was then equilibrated using 1000 μL of 100 mM ammonium acetate, pH 5.5. Vacuum-based suctioning was applied and fluids sent to waste. The prepared stock sample (1000 μL) was loaded on to the cartridge and suction was applied. A first wash of 600 μL 100 mM ammonium acetate, pH 5.5 was applied and allowed to pass through the cartridge into the waste. A second wash of 600 μL of 50% MeOH was added to the cartridge and allowed to pass therethrough into the waste. To elute the extracts from the cartridge 200 μL of one of elution solutions (i.e., eluent 1, eluent 2, or eluent 3) was added to the cartridge to elute the bound analytes into a collection tube. The particular elution solution was added a total of three time to collect a pooled volume from the cartridge. For example, 200 μL of eluent 1 was added to a cartridge a total of three times to collect a pooled volume eluted using eluent 1 (pooled sample 1). Eluent 2 (200 μL) was added to a cartridge a total of three times to collect a pooled volume eluted using eluent 2 (pooled sample 2). And 200 μL of eluent 3 was added to a cartridge a total of three time to collected a pooled volume eluted using eluent 3 (pooled sample 3).

Pooled samples (i.e., pooled sample 1, pooled sample 2, and pooled sample 3) were diluted with equal volume of 100 mM ammonium acetate and Milli-Q water and analyzed using LC-UV injection.

TABLE 7
LC-UV Data Acquisition Details

| | |
|---|---|
| UHPLC System | ACQUITY ™ PREMIER UPLC ™ |
| | (Waters Corporation) |
| Column | ACQUITY ™ PREMIER BEH C18 OST |
| | column (p/n 186009484, Waters Corporation), |
| | 2.1 × 50 mm |
| Solvent Line A | 0.1 M TEAA (100) |
| Solvent Line B | 0.1 M TEAA/Acetonitrile (50/50) |
| Col Temp | 60° C. |
| Sample Temp | 4° C. |
| Injection Vol | 5.0 μL |
| Injection type | Full loop, needle overfill |
| Weak Wash | 100% Milli-Q water (600 μL) |
| Strong Wash | 10% Acetonitrile/90% HPLC grade water |
| | (200 μL) |
| Seal Wash | 20/80 ACN/H₂O |
| Detector | TUV |
| Wavelength | 260 nm |
| Filter | None |
| Flow Rate | 0.6 mL/min |
| Sampling rate | 20 points per second |

TABLE 8
LC-UV Gradient Parameters

| Time | Flow Rate | | | |
|---|---|---|---|---|
| (min) | (mL/min) | % A | % B | Curve |
| 0.0 | 0.6 | 99.9 | 0.1 | |
| 15 | 0.6 | 0 | 100 | 6 |
| 15.1 | 0.6 | 0 | 100 | 1 |
| 18.0 | 0.6 | 99.9 | 0.1 | 1 |

Using the above procedures, recovery of analytes by the three different SPE eluents (eluent 1, eluent 2, and eluent 3) was tested. Table 9 below provides the results. Higher recoveries were observed when the SPE eluent contained TEA with concentrations exceeding 50 mM and at least 0.3% ammonium hydroxide. Irrespective of the eluent concentrations, the pH of the SPE eluent remained constant indicating that the ionic strength of TEA and ammonium hydroxide influence the recovery of the bound analytes (i.e. 20-mer ssDNA and Lipid ASO).

TABLE 9
Effect of SPE eluent composition on oligonucleotide recoveries

| Eluent | Composition of Eluent | 20-mer ssDNA | Lipid ASO |
|---|---|---|---|
| 1 | 50 mM TEA, 50% MeOH | 84.5 ± 7.4% | 72.5 ± 0.9% |
| 2 | 50 mM TEA, 50% MeOH, | 84.9 ± 7.8% | 70.0 ± 0.8% |
| | 0.15% NH₄OH | | |
| 3 | 100 mM TEA, 50% MeOH, | 93.4 ± 2.4% | 90.4 ± 0.9% |
| | 0.3% NH₄OH | | |

### Example 13—Oligonucleotide Urine and Plasma WAX SPE Recovery Using the Methods and Elution Solutions of the Present Technology on Samples Containing the Oligodeoxythymidine (15-35T), Gem91, Gem132 Oligonucleotides

In this example, the three elution solutions from example 12 are evaluated with respect to recovery of oligonucleotides using the methods of the present technology. Two different oligonucleotide samples were analyzed; a urine sample and a plasma sample. Each of the urine sample and plasma sample were spiked with 4 different oligonucleotide containing solutions: Oligodeoxythymidine ladder (15, 20, 25, 30 and 35-ODT), GEM91, GEM132, and Lipid Modified, with concentrations of 1 μg/mL final for GEM91 and 132, and 0.1 pmol/μL final for the oligodeoxythymidine and lipid modified oligonucleotides.

The spiked urine and plasma samples were each processed and prepared for use by diluting 100 μL of the spiked (plasma or urine) sample with 20 μL of 6M guanidinium hydrochloride in 60 mM pH 7.5 Tris buffer (0.66 M final), 10 μL of 500 mM tris(2-carboxyethyl) phosphine (˜28 mM final) and 50 μL proteinase K, 20 mg/mL solution (Qiagen, part number 19131). The processed samples were vortexed and subsequently incubated at 55° C. for 60 minutes.

After incubation, solid phase extraction was performed using a 96-well micro elution plate packed with 2 mg of Oasis WAX 30 μm sorbent and a vacuum manifold. Each sorbent bed was first conditioned with two 200 μL volumes of methanol and then equilibrated with two, 200 μL volumes of 50 mM ammonium acetate pH 5.5 buffer. The entire volume of the digested plasma samples (180 μL) was loaded on-top of 100 μL water pre-loaded to the SPE wells. These samples were then slowly evacuated from the SPE wells. The samples were then washed with another two 200 μL volumes of 50 mM ammonium acetate pH 5.5 buffer as well as one, 200 μL volume of 10% methanol. Finally, the adsorbed and purified analytes were eluted from the SPE wells using two, 25 μL volumes of one of the three eluent solutions (eluent: 1 comprised of 50 mM triethylamine (TEA) in 50% methanol; eluent 2: comprised of 50 mM TEA in 50% methanol with 0.15% ammonium hydroxide (NH₄OH); or eluent 3: comprised of 100 mM TEA in 50% methanol with 0.3% ammonium hydroxide (NH₄OH)).

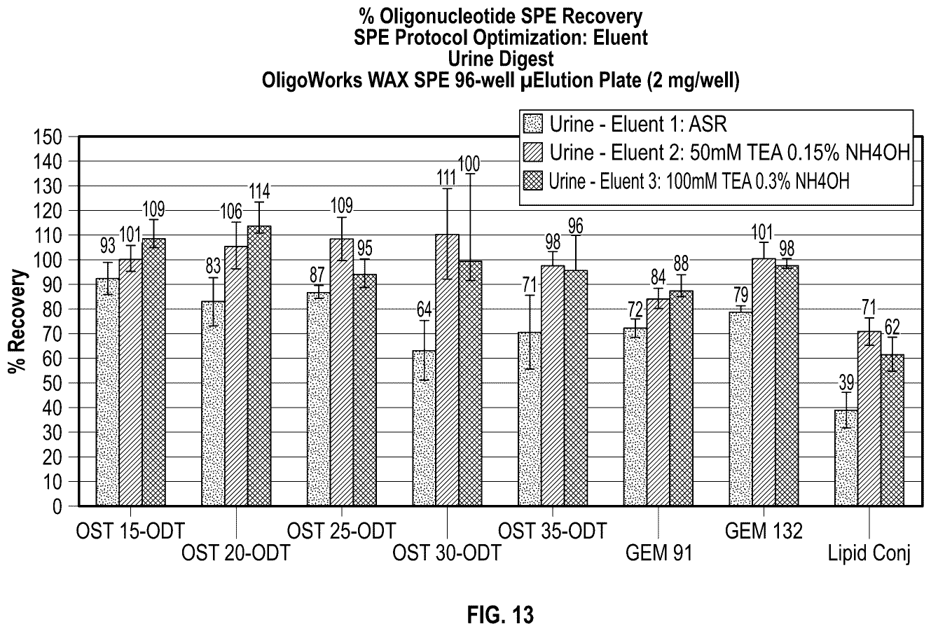

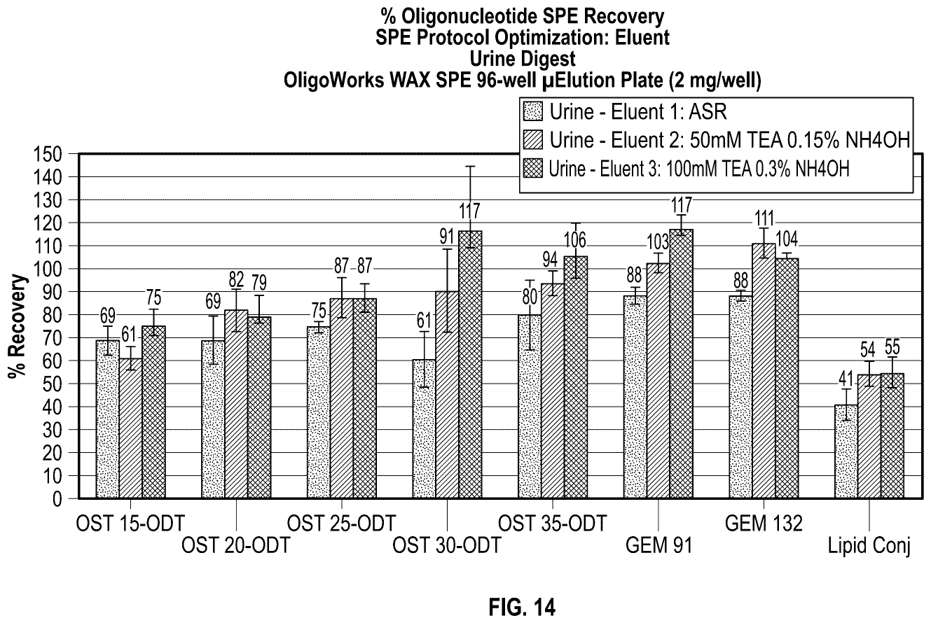

The resulting samples were diluted with an equal volume of water and then directly injected onto ion pairing reversed phase chromatography and detected by triple quadrupole mass spectrometry. That is, 6 samples were injected and analyzed by triple quadrupole mass spectrometry: urine spiked oligonucleotide eluted with eluent 1 (FIG. 13, left hand bar); urine spiked oligonucleotide eluted with eluent 2 (FIG. 13, center bar); urine spiked oligonucleotide eluted with eluent 3 (FIG. 13, right hand bar); plasma spiked oligonucleotide eluted with eluent 1 (FIG. 14, left hand bar); plasma spiked oligonucleotide eluted with eluent 2 (FIG. 14, middle bar); and plasma spiked oligonucleotide eluted with eluent 3 (FIG. 14, right hand bar).

The data shown in FIG. 13 and FIG. 14 indicates that while eluent 1 provides recovery of the oligonucleotides, eluents 2 and 3 (which include combinations of the organic amine with ammonium hydroxide) provide improved or optimized recovery, and in some instances provide almost a 1.5× increase in recovery (lipid modified results).

volume of water and then directly injected onto ion pairing reversed phase chromatography and detected by triple quadrupole mass spectrometry.

While this disclosure has been particularly shown and described with reference to example embodiments thereof, it will be understood by those skilled in the art that various changes in form and details may be made therein without departing from the scope of the technology encompassed by the appended claims.

1. A method of extracting one or more oligonucleotides from a biological sample comprising:
proteolytically digesting the sample in a detergent free mixture by combining the biological sample with a protease solution;
loading the digested sample onto a sorbent material comprising porous particles, wherein a surface of the porous particles is modified with a ligand bearing protonatable groups having a pKa value of between about 8 and about 11;
flowing one or more washing solutions through the sorbent material such that one or more non-oligonucleotide components are removed from the sorbent material while the one or more oligonucleotides are retained on the sorbent material; and
flowing an elution solution having a pH ranging from about 10 to about 12 through the sorbent material such that the retained one or more oligonucleotides are released from the sorbent material into the elution solution, resulting in an eluate.

2. The method of claim 1, wherein the protease solution comprises proteinase K.

3. The method of claim 1, wherein the detergent free mixture further comprises guanidine.

4. The method of claim 1, wherein the porous particles have a size greater than 5 μm and less than 100 μm.

5. The method of claim 1, wherein the method further comprises subjecting the eluate to liquid chromatography, mass spectrometry (MS), ultraviolet-visible spectroscopy, or combinations thereof.

6. The method of claim 1, wherein the method further comprises directly injecting the eluate, without evaporation, into an ion pairing reversed phase separation.

7. The method of claim 1, wherein the one or more washing solutions comprises a partially aqueous organic solvent solution comprising methanol or ethanol, combinations thereof.

8. The method of claim 1, wherein the elution solution comprises a base selected from organic amine, ammonium bicarbonate, ammonium hydroxide, ammonium acetate, or combinations thereof.

9. The method of claim 1, wherein the one or more elution solutions comprise the organic amine triethylamine (TEA).

10. The method of claim 1, wherein the biological sample comprises a biological fluid selected from the group consisting of whole blood samples, blood plasma samples, serum samples, oral fluids, cerebrospinal fluids, fecal samples, nasal samples, and urine.

11. The method of claim 1, wherein the biological sample comprises a biological tissue selected from the group consisting of liver, kidney and brain tissue, tissue homogenates, cells, and cell culture supernatants.

12. The method of claim 11, wherein the biological sample comprises tissue homogenates, and a surfactant free tissue homogenization step is performed simultaneously with proteolytically digesting the sample.

13. The method of claim 11, wherein the biological sample comprises tissue homogenates, and a surfactant free tissue homogenization step is performed prior to proteolytically digesting the sample.

14. The method of claim 12, wherein an organic solvent is added with a concentration of greater than 20% volume (e.g., 50% or more) during the tissue homogenization step.

15. The method of claim 13, wherein after the tissue homogenization step, the tissue homogenate is diluted two to four fold with an aqueous solution and then incubated with a protease for proteolytically digesting the sample.

16. The method of claim 1, wherein the one or more oligonucleotides are selected from double-stranded RNA, single-stranded RNA, single stranded DNA, double stranded DNA, double standard RNA/DNA hybrid, synthetic RNA, synthetic DNA, and combinations thereof, wherein the one or more nucleotides have a size ranging from a 10 mer to a 200 mer.

17. The method of claim 1, wherein the eluent solution further comprises an organic solvent at a concentration between 10 and 70% volume.

18. The method of claim 1, further comprising diluting the eluate with an equal volume of water.

19. The method of claim 1, wherein the protease solution, the sorbent material, and the elution solution are comprised in a kit.

20. A method of extracting one or more oligonucleotides from a biological sample comprising:
proteolytically digesting the sample by combining the biological sample with a proteinase K solution and guanidine;
loading the digested sample onto a sorbent material comprising porous particles, wherein the surface of the porous particles is modified with a ligand bearing protonatable groups having a pKa value of between about 8 and about 11, (e.g., between about 8 and about 10);
flowing one or more washing solutions through the sorbent material such that one or more non-oligonucleotides components are removed from the sorbent material while the one or more oligonucleotides are retained on the sorbent material; and
flowing an elution solution having a pH ranging from about 10 to about 12 through the sorbent material such that the retained one or more oligonucleotides are released from the sorbent material into the elution solution, resulting in an eluate.
